(19) United States
(12) Patent Application Publication
(10) Pub. No.: US 2026/0274788 A1
(43) Pub. Date: Sep. 17, 2026

(54) PROCESS FOR PREPARING (R)-2-AMINO-3-PHENYLPROPYL CARBAMATE

(71) Applicant: INKE, S.A., Barcelona (ES)

(72) Inventors: Daniel ALONSO DÍAZ, Barcelona (ES); Sergio RODRIGUEZ ROPERO, Barcelona (ES)

(21) Appl. No.: 19/472,110

(22) PCT Filed: Apr. 2, 2024
(86) PCT No.: PCT/EP2024/058857

§ 371 Date: Oct. 3, 2025

(30) Foreign Application Priority Data
Apr. 3, 2023 (EP) 23382319.4

(51) Int. Cl. C07C 269/06; C07C 271/12

(52) U.S. Cl. CPC C07C 269/06; C07C 271/12

(57) ABSTRACT

The present invention relates to an improved process for preparing (R)-2-amino-3-phenylpropyl carbamate in high yield and high purity. Particularly, the process involves the carbamoylation of a nitrogen-protected (R)-2-amino-3-phenylpropan-1-ol, under specific reaction conditions to provide (R)-2-amino-3-phenylpropyl carbamate having simultaneously low content of major impurities A and B, without the need of further purification steps.

### FIELD OF THE INVENTION

The present invention relates to an improved process for preparing (R)-2-amino-3-phenylpropyl carbamate, or a pharmaceutically acceptable acid addition salt thereof, in high yield and high purity. Particularly, the process involves the carbamoylation of a nitrogen-protected (R)-2-amino-3-phenylpropan-1-ol, under specific reaction conditions to provide (R)-2-amino-3-phenylpropyl carbamate, or a pharmaceutically acceptable acid addition salt thereof, having simultaneously low content of major impurities A and

B, without the need of further purification steps.

### BACKGROUND OF THE INVENTION

(R)-2-amino-3-phenylpropyl carbamate, also known as solriamfetol, compound of formula (I), is a phenylalanine derivative and an atypical wake-promoting drug. Solriamfetol indirectly enhances dopaminergic and noradrenergic neurotransmission, and it does not inhibit serotonin reuptake, release monoamines, or inhibit monoamine oxidase A (MAO-A) enzymatic activity. Solriamfetol hydrochloride is approved as film coated tablets of 75 mg and 150 mg strengths under the tradename SUNOSI.

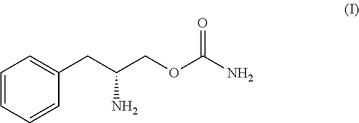 [SMILES: NC(=O)OC[C@H](N)Cc1ccccc1]

SUNOSI is used to improve wakefulness and reduce excessive daytime sleepiness in adults with narcolepsy or obstructive sleep apnoea. Narcolepsy is a long-term sleep disorder, which affects the brain's ability to regulate the normal sleep-wake cycle. This leads to symptoms such as an irresistible urge to sleep, even at inappropriate times and places, and disturbed night-time sleep. SUNOSI is used in patients with or without cataplexy (episodes of severe muscle weakness that can cause collapse). Obstructive sleep apnoea is repeated interruption of breathing during sleep due to airways becoming blocked. SUNOSI is used when other treatments, such as continuous positive airway pressure (CPAP, use of a ventilator to keep the airways open), have not satisfactorily improved the excessive daytime sleepiness.

WO096/24577 A discloses the synthesis of solriamfetol by reacting (R)-2-amino-3-phenylpropan-1-ol, also named (D)-phenylalaninol, with benzyl chloroformate in a basic aqueous solution to give N-benzyloxycarbonyl-(D)-phenylalaninol, which is subjected to carbamoylation with phosgene in the presence of an amine base to provide O-carbamoyl-N-benzyloxycarbonyl-(D)-phenylalaninol, followed by an hydrogenolysis in the presence of a catalyst, and further reacted with an anhydrous hydrochloric acid to give the corresponding hydrochloride salt. This process uses phosgene which is an extremely toxic reagent by acute (short-term) inhalation exposure, causing severe respiratory effects, including pulmonary edema, pulmonary emphysema, and death have been reported in humans and severe ocular irritation and dermal burns may result following eye or skin exposure.

WO2005/033064 A1 discloses a process for preparing solriamfetol via a chemoselective carbamoylation of a hydroxyl group in the presence of a more reactive amine group in a single step by using a cyanate and an excess of acid in an organic medium. Particularly, (D)-phenylalaninol is reacted with sodium cyanate and an excess of methane sulfonic acid in dichloromethane to provide solriamfetol in 89% yield. Impurities are not disclosed.

WO2020/035769 A1 discloses a purification process of solriamfetol consisting of reacting crude solriamfetol with an organic acid and an optically active acid to form the corresponding solriamfetol acid addition salt, followed by converting the solriamfetol acid addition salt to solriamfetol hydrochloride. According to this purification process, one or more of impurity A, impurity B, impurity C, impurity D, impurity E, impurity F, impurity G, impurity H or impurity I are present in less than 0.15% w/w relative to the amount of solriamfetol hydrochloride as determined by HPLC.

WO2021/250067 A2 discloses a purification process of solriamfetol via salt formation, wherein crude solriamfetol is reacted with an aliphatic acid or aromatic monocarboxylic acid, followed by converting the solriamfetol acid addition salt to (R)-2-amino-3-phenylpropyl carbamate or its hydrochloride salt having impurities such as phenylalaninol (Impurity 1), (R)-2-amino-3-phenylpropyl (aminocarbamoyl) carbamate (Impurity 3), and N-1-hydroxymethyl-2-phenyl-ethyl-N′-aminocarbamoyl urea (Impurity 5), at a concentration less than about 0.15% by weight.

Therefore, from what is known in the art, there is a need for an efficient and robust process for the preparation of (R)-2-amino-3-phenylpropyl carbamate, which avoids the formation of process-related impurities, while affording the desired solriamfetol hydrochloride product with high yield and high purity.

### SUMMARY OF THE INVENTION

Prior art processes involving the carbamoylation of (R)-2-amino-3-phenylpropan-1-ol or of nitrogen-protected (R)-2-amino-3-phenylpropan-1-ol thereof, provide solriamfetol having as main impurities the unreacted starting material (R)-2-amino-3-phenylpropan-1-ol (Impurity A) and the (R)-(2-amino-3-phenylpropyl)-N-carbamoylcarbamate (Impurity B), which derives from a second addition of the carbamoyl function to the previously formed (R)-2-amino-3-phenylpropyl carbamate skeleton.

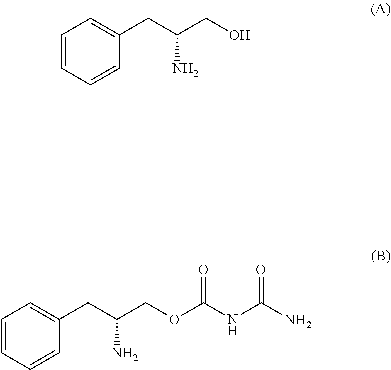 [SMILES: N[C@@H](CO)Cc1ccccc1 ; NC(=O)NC(=O)OC[C@H](N)Cc1ccccc1]

When nitrogen-protected (R)-2-amino-3-phenylpropan-1-ol is used, at the end of the carbamoylation reaction, the reaction mixture contains two major impurities, i.e., impurity (III) and impurity (V), which are the precursors of impurities A and B, respectively, wherein the amino groups are substituted with the protecting group P used in the manufacturing process.

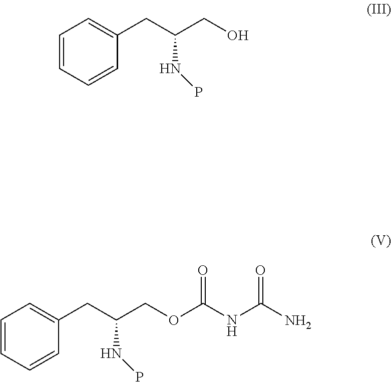

When the carbamoylation reaction is carried out with non-protected nitrogen-(R)-2-amino-3-phenylpropan-1-ol, impurities (A) and (B) are detected in the resulting reaction mixture. The presence of impurities (A) and (B) or impurities (III) and (V) are strictly correlated, because the attempt to push the conversion to completeness by using an excess of reagents, allows a complete consumption of the starting material, i.e. impurity (A) or (III) but inevitably has the consequence of increasing the content of impurity (B) or (V). On the other hand, decreasing the excess of the reagents to limit the formation of impurity (B) or (V) generally leads to larger amount of residual starting material impurity (A) or (III).

The present inventors have surprisingly found specific carbamoylation reaction conditions for the preparation of (R)-2-amino-3-phenylpropyl carbamate having simultaneously starting material impurity (A) and impurity (B) below 0.15% w/w as determined by HPLC method B without the need of further purification steps, such as salt formation with an organic acid, thereby allowing a more cost-effective obtention of (R)-2-amino-3-phenylpropyl carbamate, i.e. compound of formula (I).

Therefore, a first aspect of the present invention relates to a process for preparing (R)-2-amino-3-phenylpropyl carbamate or a pharmaceutically acceptable acid addition salt thereof, the process comprising the steps of:
a) providing a mixture comprising nitrogen-protected (R)-2-amino-3-phenylpropan-1-ol, compound of formula (III)

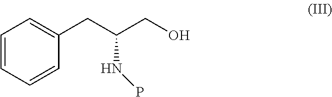

wherein P is a N-protecting group selected from carbobenzyloxy (Cbz), p-methoxybenzyl (PMB), and 3,4-dimethoxybenzyl (DMPM), and an alkali metal cyanate in an amount of 1.0 to 1.8, preferably of 1.0 to 1.6, more preferably of 1.1 to 1.5, even more preferably of 1.3 to 1.35 molar equivalents with respect to compound of formula (III), in the presence of a solvent,
b) adding an acid with a pKa measured in water at 25° C. lower than 1.0, in an amount of 1.5 to 3.5, preferably of 1.8 to 2.5, more preferably of 2.0 to 2.35, even more preferably of 2.25 to 2.35 molar equivalents with respect to the alkali metal cyanate to the mixture of step (a), at a temperature from 0° C. to 10° C., to provide O-carbamoyl-N-protected-(R)-2-amino-3-phenylpropan-1-ol, compound of formula (II), wherein P is as defined above,

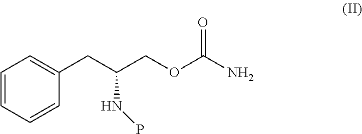

c) adding at least one portion of an alkali metal cyanate wherein each portion is used in an amount of 0.1 to 0.3 molar equivalents with respect to compound of formula (III) to the mixture of step (b), at a temperature from 0° C. to 10° C.,
d) removing the N-protecting group from the compound of formula (II) to give O-carbamoyl-(R)-2-amino-3-phenylpropan-1-ol, compound of formula (I),

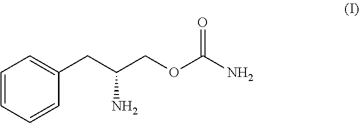 [SMILES: NC(=O)OC[C@H](N)Cc1ccccc1]

e) optionally, converting the compound of formula (I) into a pharmaceutically acceptable acid addition salt thereof.

A second aspect of the invention refers to (R)-2-amino-3-phenylpropyl carbamate or a pharmaceutically acceptable salt thereof, preferably the hydrochloride acid addition salt, obtainable by the process according to the invention having simultaneously (R)-2-amino-3-phenylpropan-1-ol (impurity A) and the (R)-(2-amino-3-phenylpropyl)-N-carbamoylcarbamate (impurity B) in an amount below 0.15% w/w as measured by HPLC method B.

A third aspect of the invention refers to pharmaceutical compositions comprising (R)-2-amino-3-phenylpropyl carbamate as defined in the second aspect and one or more pharmaceutically acceptable carriers, diluents or excipients.

A fourth aspect of the invention refers to the use (R)-2-amino-3-phenylpropyl carbamate as defined in the second aspect or the use of the pharmaceutical compositions as defined in the third aspect in the treatment of narcolepsy and obstructive sleep apnoea.

### Definitions

When describing the compounds and methods of the invention, the following terms have the following meanings, unless otherwise indicated.

The term “about” as used herein refers to a statistically meaningful range of a value, typically within 10%. Such a range can lie within experimental error, typical of standard methods used for the measurement and/or determination of a given value or range. In one embodiment, the range is within 5% of the indicated value. In another embodiment, the range is within 1% of the indicated value. In yet another embodiment, the range is within 0.5% of the indicated value.

As used herein, the term “solvent” refers to water or an organic molecule capable of at least partially dissolving another substance (i.e., the solute). Solvents may be liquids at room temperature. Suitable solvents may be, but are not limited to (C₆-C₁₄) aromatic hydrocarbon solvents such as toluene, o-xylene, m-xylene, and p-xylene; halogenated (C₁-C₁₂) hydrocarbon solvents such as 1,2-dichloroethane, dichloromethane, chloroform; (C₁-C₁₂) ether solvents such as diethyl ether, dipropyl ether, diphenyl ether, isopropyl ether, tert-butyl methyl ether, tetrahydrofuran, 1,4-dioxane; (C₁-C₁₂) ester solvents such as ethyl formate, methyl acetate, ethyl acetate, isopropyl acetate, butyl acetate, isobutyl acetate, ethyl malonate; (C₃-C₁₂) ketone solvents such as acetone, methyl ethyl ketone or 2-butanone, methyl isobutyl ketone, cyclohexanone, cyclopentanone, 3-pentanon; (C₁-C₁₂) alcohol solvents such as methanol, ethanol, isopropanol, 1-propanol, 2-methyl-1-propanol, 1-butanol, 2-butanol, 1-pentanol, 3-methyl-1-butanol, tert-butanol, 1-octanol, benzylalcohol, phenol, trifluoroethanol, glycerol, ethylene glycol, propylene glycol, m-cresol; nitrobenzene; N,N-dimethylformamide; N,N,-dimethylacetamide; N-methyl-2-pyrrolidone; or acetonitrile. In another embodiment, the solvent is formed by the combination of two or more solvents.

As used herein, the term “alcohol” refers to a hydrocarbon derivative in which one or more hydrogen atoms have been replaced by an —OH group, known as hydroxyl group.

Suitable alcohols include linear, cyclic or branched C₁-C₆ alkyl alcohols and any mixtures thereof. It also includes commercially available alcohols. In another embodiment, the alcohol is methanol, ethanol, isopropanol, 1-propanol and 1-butanol, and mixtures thereof.

As used herein, the term “room temperature” in the context of the present invention refers to a temperature from 15° C. to 30° C., preferably from 20° C. to 25° C.

As used herein, a “one-pot” or “consecutive manner” process designates a process in which there are at least two reaction steps carried out without isolation and/or purification of the intermediate product or products, and suitably carried out in a single reaction vessel/container. It will be understood by one of skill in the art that a simple transfer of the whole reaction mass at an intermediate stage, but without isolating and/or purifying the intermediate product, is still a “one-pot” process or “consecutive manner” according to the present invention, not the least because such a process would still achieve the technical advantage associated with a one-pot process in that the intermediate formed in situ does not need to be isolated and/or purified.

As used herein, the term “solvent extraction” refers to the process of separating components of a mixture by using a solvent which possesses greater affinity for one component and may, therefore, separate said one component from at least a second component which is less miscible than said one component with said solvent.

The term “filtration” refers to the act of removing solid particles greater than a predetermined size from a feed comprising a mixture of solid particles and liquid. The expression filtrate refers to the mixture less the solid particles removed by the filtration process. It will be appreciated that this mixture may contain solid particles smaller than the predetermined particle size. The expression filter cake refers to residual solid material remaining on a feed side of a filtration element.

The term “evaporation” refers to the change in state of solvent from liquid to gas and removal of that gas from the reactor. Various solvents may be evaporated during the synthetic route disclosed herein. As known to those of skilled in the art, each solvent may have a different evaporation time and/or temperature.

The term “phase separation” refers to a solution or mixture having at least two physically distinct regions.

The term “crystallization” refers to any method known to a person skilled in the art such as crystallization from single solvent or combination of solvents by dissolving the compound, optionally at elevated temperature and precipitating the compound by cooling the solution or removing solvent from the solution or both. It further includes methods such as dissolving the compound in a solvent and precipitating it by addition of an “antisolvent” (i.e. a solvent in which the desired compound has low solubility or insolubility, and can be used to precipitate such compound by adding it to a solution in which the compound is dissolved).

The terms “conventional isolation techniques” or “purification” as used herein refers to the process of rendering a product clean of foreign elements whereby a purified product can be obtained. The term industrial purification refers to purifications, which can be carried out on an industrial scale such as solvent extraction, filtration, slurring, washing, phase separation, distillation, centrifugation or crystallization.

The term “pharmaceutically acceptable salt” refers to a salt prepared from an acid, which is acceptable for administration to a patient, such as a mammal. Such salts can be derived from pharmaceutically acceptable inorganic or organic acids. Suitable inorganic acid is selected from the group consisting of hydrochloric acid, hydrobromic acid, sulfuric acid and phosphoric acid. Suitable organic acid is selected from the group consisting of formic acid, acetic acid, trifluoroacetic acid, trichloroacetic acid, benzoic acid, citric acid, malonic acid, salicylic acid, malic acid, fumaric acid, oxalic acid, succinic acid, tartaric acid, lactic acid, gluconic acid, ascorbic acid, maleic acid, pamoic acid, aspartic acid, benzene sulfonic acid, methane sulfonic acid, ethane sulfonic acid, hydroxymethane sulfonic acid and hydroxyethane sulfonic acid. Preferably, the pharmaceutically acceptable salt of (R)-2-amino-3-phenylpropyl carbamate is the hydrochloride salt.

### DETAILED DESCRIPTION OF THE INVENTION

According to the first aspect, the present invention relates to a process for preparing (R)-2-amino-3-phenylpropyl carbamate or a pharmaceutically acceptable acid addition salt thereof having simultaneously the unreacted starting material (R)-2-amino-3-phenylpropan-1-ol (impurity A) and the (R)-(2-amino-3-phenylpropyl)-N-carbamoylcarbamate (impurity B) below 0.15% w/w as determined by HPLC method B.

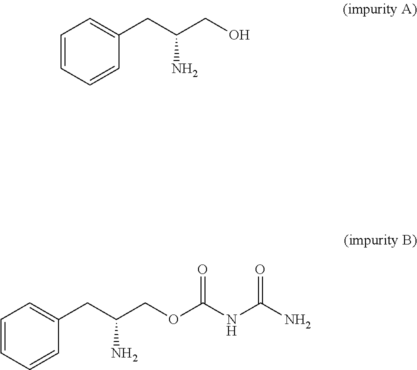 [SMILES: N[C@@H](CO)Cc1ccccc1 ; NC(=O)NC(=O)OC[C@H](N)Cc1ccccc1]

In certain embodiments, the process of the present invention provides the (R)-2-amino-3-phenylpropyl carbamate or a pharmaceutically acceptable salt thereof, preferably the hydrochloride acid addition salt, having simultaneously impurities A and B below 0.15% w/w as measured by HPLC method B.

In certain embodiments, the alkali metal cyanate of steps (a) and (c) is selected from the group consisting of sodium cyanate and potassium cyanate, and mixtures thereof, preferably the alkali metal cyanate of steps (a) and (c) is sodium cyanate.

Suitable solvent used in step (a) may be a halogenated solvent selected from the group consisting of dichloromethane, 1,2-dichloroethane, chloroform, and chlorobenzene, and mixtures thereof, preferably the solvent of step (a) is dichloromethane.

Suitable acid of step (b) has a pKa measured in water at 25° C. lower than 1.0 and may be selected from the group consisting of hydrochloric acid, hydrobromic acid and methane sulfonic acid and mixtures thereof, preferably the acid with a pKa measured in water at 25° C. lower than 1.0 is methane sulfonic acid.

In an embodiment, the temperature of steps (a) and (b) are from 0° C. to 10° C., preferably from 0° C. to 5° C. In an embodiment, the resulting mixture of step (b) may be stirred at a temperature for about 1 hour, about 2 hours, about 3 hours, about 4 hours, or about 5 hours. In another embodiment, the resulting mixture of step (b) is stirred at a temperature from 0° C. to 10° C. for about 4 hours before proceeding with step (c).

Advantageously, adding at least one portion of an alkali metal cyanate wherein each portion is used in an amount of 0.1 to 0.3 molar equivalents with respect to compound of formula (III), wherein P is as defined above, to the mixture of step (b), at a temperature from 0° C. to 10° C., allows to complete consumption of the starting material while maintaining the amount of impurity (V) below 0.15% w/w as measured by HPLC method A. This also allows to obtain a final product, after completion of steps (d) and optionally (e), having an amount of both (R)-2-amino-3-phenylpropan-1-ol (impurity A) and (R)-(2-amino-3-phenylpropyl)-N-carbamoylcarbamate (impurity B) below 0.15% w/w as measured by HPLC method B without the need of further purification steps.

In an embodiment, the mixture of step (c) is stirred at a temperature of from 0° C. to 10° C., preferably from 5° C. to 10° C., for at least about 2 hours, preferably for about 4 hours.

In an embodiment, one portion of an alkali metal cyanate is added in step (c) in an amount of 0.1 to 0.3 molar equivalents with respect to compound of formula (III), wherein P is as defined above, at a temperature from 0° C. to 10° C., preferably from 5° C. to 10° C., and the resulting mixture is stirred for at least about 2 hours, preferably about 4 hours.

In an embodiment, a second portion of an alkali metal cyanate is added in step (c) in an amount of 0.1 to 0.3 molar equivalents with respect to compound of formula (III), wherein P is as defined above, at a temperature from 0° C. to 10° C., preferably from 5° C. to 10° C., and the resulting mixture is stirred for at least about 2 hours, preferably about 4 hours.

In an embodiment, the resulting mixture of step (c) may be further stirred at a temperature of from 15° C. to 25° C. for about 6 hours, about 8 hours, about 12 hours, or about 24 hours.

In an embodiment, the process further comprises adding water and an alkali metal hydroxide, preferably sodium hydroxide, to adjust the pH between 9 to 11, at a temperature from 0° C. to 10° C., to the mixture obtained after step (c) and before carrying out step (d).

In an embodiment, compound of formula II, wherein P is as defined above, obtained after step (c) is isolated and purified by crystallization from an alcohol selected from methanol, ethanol, isopropanol and 1-propanol, and mixtures thereof, preferably isopropanol. In an embodiment, the compound of formula II wherein R is benzyl, e.g., benzyl (R)-(1-hydroxy-3-phenylpropan-2-yl) carbamate, obtained after step (c) is isolated and recrystallized from an alcohol selected from methanol, ethanol, isopropanol and 1-propanol, and mixtures thereof, preferably isopropanol.

In an embodiment, the N-protecting group of compound of formula II is removed in step (d) through hydrogenolysis in the presence of a catalyst and a solvent. Suitable catalyst may be selected from the group consisting of Pd, Pt, Rh, Ru, Ni, Fe, Zn and Ir catalyst, preferably palladium (0), palladium hydroxide (Pd(OH)₂), palladium on activated carbon (Pd/C), palladium on alumina, palladium on carbon powder, platinum, platinum on activated carbon and Raney nickel. In an embodiment, the amount of catalyst used in step (d) may be from 1% to 15%, preferably from 1% to 10%, more preferably from 1% to 5% w/w with respect to the compound of formula II.

Suitable solvent used in step (d) may be selected from an ether, an alcohol and an ester solvent, preferably the ether solvent is selected from the group consisting of diethyl ether, diisopropyl ether, diphenyl ether, tetrahydrofuran, 2-methyltetrahydrofuran and 1,4-dioxane, the alcohol solvent is selected from the group consisting of methanol, ethanol, isopropanol and 1-propanol, and the ester solvent is selected from the group consisting of methyl acetate, ethyl acetate and isopropyl acetate.

In another embodiment, compound of formula II is benzyl (R)-(1-(carbamoyloxy)-3-phenylpropan-2-yl) carbamate and the benzyl protecting group is removed by hydrogenolysis in the presence of 1% to 10% w/w of palladium on carbon in the presence of an ether solvent selected from the group consisting of diethyl ether, diisopropylether, diphenylether, tetrahydrofuran, 2-methyltetrahydrofuran and 1,4-dioxane, preferably tetrahydrofuran.

In certain embodiments, step (e) is carried out and compound of formula I is converted into a pharmaceutically acceptable acid addition salt, preferably the hydrochloride acid addition salt. In an embodiment, compound of formula I obtained after step (d) is converted into the pharmaceutically acceptable acid addition salt, preferably the hydrochloride acid addition salt, without purification. In another embodiment, steps (d) and (e) are carried out in a consecutive manner, i.e. in one-pot manner, wherein compound (I) thus obtained is not isolated, as depicted in Scheme 1.

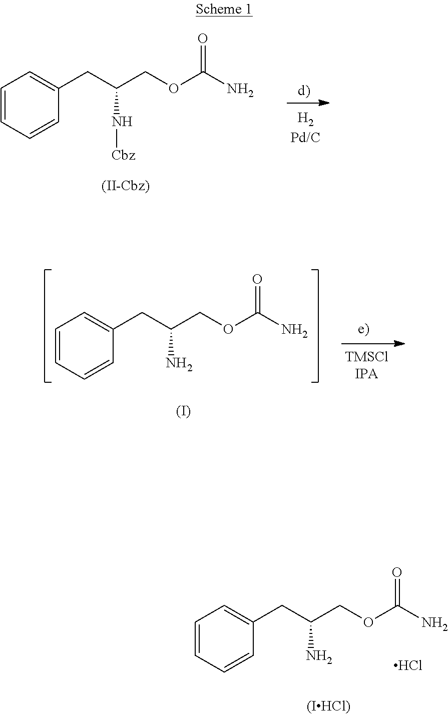

In an embodiment, compound of formula I is converted into the hydrochloride acid addition salt in anhydrous conditions. In an embodiment, compound of formula I may be reacted with trimethylsilyl chloride in the presence of an alcohol selected from methanol, ethanol, 2-propanol or isopropanol and 1-propanol, and mixtures thereof. In an embodiment, the amount of trimethylsilyl chloride is of 1.0 to 2.0 molar equivalents with respect to compound of formula I. In an embodiment, the hydrochloride salt formation of compound of formula I may be carried out at a temperature of from 0° C. to 10° C., preferably from 0° C. to 5° C.

In an embodiment, compound of formula I, i.e. (R)-2-amino-3-phenylpropyl carbamate, is reacted with trimethylsilyl chloride in an amount of 1.0 to 2.0 molar equivalents in the presence of isopropanol at a temperature of from 0° C. to 10° C. In an embodiment, the polymorphic form of (R)-2-amino-3-phenylpropyl carbamate hydrochloride obtained is the known anhydrous Form A.

In certain embodiments, intermediate nitrogen-protected (R)-2-amino-3-phenylpropan-1-ol, compound of formula (III) of step (a), is prepared by a process which comprises the steps of:
i. providing (R)-2-amino-3-phenylpropan-1-ol in a mixture of a ketone solvent and water in a ratio of 6:1 to 4:1, preferably 5:1,
ii. adding an inorganic base selected from an alkali metal carbonates and bicarbonates,
iii adding a N-protecting group selected from the group consisting of benzyl-(2,5-dioxopyrrolidin-1-yl) carbonate, phenylchloroformate, p-methoxybenzyl (PMB), and 3,4-dimethoxybenzyl (DMPM), preferably benzyl-(2,5-dioxopyrrolidin-1-yl) carbonate, and
iv. optionally, isolating the corresponding N-protected-(R)-2-amino-3-phenylpropan-1-ol, compound of formula (III), wherein P is as defined above.

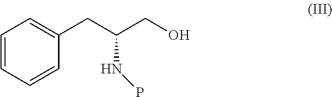

Suitable ketone solvent may be acetone, methyl ethyl ketone or 2-butanone, methyl isobutyl ketone, cyclohexanone, cyclopentanone, 3-pentanone, and mixtures thereof. Preferably, the ketone solvent used in step (i) is acetone. In an embodiment, the solvent of step (i) is a mixture of acetone and water in a ratio of 6:1 to 4:1, preferably 5:1.

Suitable alkali metal carbonates of step (ii) may be sodium carbonate, potassium carbonate, and alkali metal bicarbonates may be sodium bicarbonate and potassium bicarbonate, and mixtures thereof. In an embodiment, the amount of inorganic base used in step (ii) is from 2.0 to 3.0 molar equivalents with respect to the (R)-2-amino-3-phenylpropan-1-ol.

Suitable N-protecting group of step (iii) may be selected from the group consisting of benzyl-(2,5-dioxopyrrolidin-1-yl) carbonate, phenylchloroformate, p-methoxybenzyl (PMB), and 3,4-dimethoxybenzyl (DMPM), preferably the N-protecting group of step (iii) is benzyl-(2,5-dioxopyrrolidin-1-yl) carbonate. In an embodiment, the amount of N-protecting group used in step (iii) is from 1.0 to 2.0, preferably is from 1.0 to 1.2 molar equivalents with respect to the (R)-2-amino-3-phenylpropan-1-ol. In an embodiment, the N-protecting group is added in one portion, in two portions, in three portions, in four portions, in five portions and in six portions.

In certain embodiments, the temperature of steps (i), (ii) and (iii) is from 0° C. to 10° C., preferably from 0° C. to 5° C.

In certain embodiments, step (iv) is carried out and compound of formula (III) is isolated by removing the solvent of step (i) and adding an aromatic hydrocarbon solvent such as toluene to crystallize the compound of formula III. In certain embodiments, the solvent mixture of step (i) is removed by co-distillation with the aromatic hydrocarbon solvent.

In the following, the present invention is further illustrated by examples. They should in no case be interpreted as a limitation of the scope of the invention as defined in the claims. Unless indicated otherwise, all indications of percentage are by weight and temperatures are in degrees Celsius.

### General Methods

HPLC Method a for Compound of Formula II, Wherein R is Benzyl, e.g. Benzyl (R)-(1-(carbamoyloxy)-3-phenylpropan-2-yl) carbamate
Column: YMC-Pack ODS AQ; octadecyl silane silica gel for chromatography (5 μm)
size 4.6 mm×25 cm
Flow: 1.2 mL/min
Wavelength: 210 nm
Column Temperature: 25° C.
Injection volume: 5 μL
Mobile phase A: 10 mM KH₂PO₄; PH=3.0 (H₃PO₄)
Mobile phase B: acetonitrile
Elution: Gradient (Table 1)

TABLE 1

| Time (min) | Mobile phase A (% V/V) | Mobile phase B (% V/V) |
|---|---|---|
| 0-2 | 60 | 40 |
| 2-31 | 60→25 | 40→75 |
| 31-31.10 | 25→60 | 75→40 |

Analysis time: 31.10 min
Retention time (RRT):
Impurity (V, wherein P is benzyl): 0.96
Impurity (III, wherein P is benzyl): 0.98
benzyl (R)-(1-(carbamoyloxy)-3-phenylpropan-2-yl) carbamate: 1.00
   HPLC method B for (R)-2-amino-3-phenylpropyl Carbamate Hydrochloride
Column: YMC-Pack ODS AQ; octadecyl silane silica gel for chromatography (5 μm)
size 4.6 mm×25 cm
Flow: 1.2 mL/min
Wavelength: 210 nm
Column Temperature: 25° C.
Injection volume: 5 μL
Mobile phase A: 10 mM KH₂PO₄; PH=3.0 (H₃PO₄)
Mobile phase B: acetonitrile
Elution: Gradient (Table 2)

TABLE 2

| Time (min) | Mobile phase A (% V/V) | Mobile phase B (% V/V) |
|---|---|---|
| 0-10 | 90→77 | 10→23 |
| 10-20 | 77→25 | 23→75 |
| 20-25 | 25 | 75 |
| 25-25.10 | 25→90 | 75→10 |

Analysis time: 25.10 min
Retention time (RRT):
Impurity (A): 0.89
Impurity (B): 2.07
benzyl (R)-(1-(carbamoyloxy)-3-phenylpropan-2-yl) carbamate: 3.10

### Differential Scanning Calorimetry (DSC)

Sample preparation: Approximately 1-4 mg of sample were weighed (using a MX5 Mettler Toledo microbalance) into 40 μL aluminium crucibles with a pinhole lid.
Data acquisition: DSC analyses were recorded in a Mettler Toledo DSC822e calorimeter. Programs used: Data collection and evaluation with software STARe.
Measurement conditions: The samples were heated under dry nitrogen (flow rate: 50 mL/min) at 10° C./min from 30 to 300° C.

### EXAMPLES

### Example 1: Preparation of benzyl (R)-(1-hydroxy-3-phenylpropan-2-yl) carbamate (Compound of Formula III Wherein P is -Cbz)

To a stirred solution of (R)-2-amino-3-phenylpropan-1-ol (100 g, 0.661 mol) in 0.5 L of acetone and 0.1 L of water cooled at 0-5° C., sodium bicarbonate (139 g, 1.655 mol) were added in two portions. Afterwards, benzyl-(2,5-dioxopyrrolidin-1-yl) carbonate (168 g, 0.674 mol) were added in four portions at the same temperature and allowed to react for 1 hour. After warming the suspension at 20-25° C., 2.0 L of toluene and 1.0 L of water were added. The resulting mixture was heated up to 40-45° C. and the phases were allowed to separate. The organic layer was washed several times with water and concentrated to induce precipitation. The solid was filtered off, washed with toluene and dried.

Yield: 162 g (86% yield)

DSC: Sharp endotherm with an onset at 94.1° C.

HPLC: 99.9%

### Example 2: Preparation of benzyl (R)-(1-hydroxy-3-phenylpropan-2-yl) carbamate (Compound of Formula III Wherein P is -Cbz)

To a stirred solution of (R)-2-amino-3-phenylpropan-1-ol (50 g, 0.331 mol) in 1.0 L of acetone and 0.2 L of water maintained at 0-5° C., sodium bicarbonate (69.5 g, 0.828 mol) were added. Afterwards, benzyl-(2,5-dioxopyrrolidin-1-yl) carbonate (84 g, 0.337 mol) were added in four portions at the same temperature and allowed to react for 1 h. 1.0 L of toluene was added after warming the suspension at 20-25° C. and then acetone was removed by distillation. Water (0.5 L) was added, and the resulting mixture stirred at 45-50° C. and the phases allowed to separate. The organic layer was washed several times with water and concentrated to induce precipitation. The solid was filtered off, washed with toluene and dried.

Yield: 83.6 g (89% yield)

DSC: Sharp endotherm with an onset at 94.1° C.

HPLC: 99.9%

### Example 3: Preparation of benzyl (R)-(1-(carbamoyloxy)-3-phenylpropan-2-yl) Carbamate (Compound of Formula II Wherein P is -Cbz)

To a stirred suspension of benzyl (R)-(1-hydroxy-3-phenylpropan-2-yl) carbamate (100 g, 0.350 mol) and sodium cyanate (33.9 g, 0.522 mol) in 2.5 L of methylene chloride at 3-5° C., methane sulfonic acid (68.2 mL, 1.051 mmol) were added dropwise. The temperature was set at 7-8° C. and the reaction allowed to react overnight. Afterwards, an extra amount of sodium cyanate (2.3 g, 0.035 mol) was added and the reaction continued for another 24 h. To the resulting mixture water (970 mL) was added and the pH adjusted to 10-11 by careful addition of 20% aqueous NaOH, controlling the temperature below 10° C., thus affording a biphasic solution. The organic phase was washed with water (2.5 L) and the solvent was partially distilled. Then 1.6 L of 2-propanol were added and distillation continued until all the methylene chloride was removed. After stirring, the suspension was filtered off, washed with cold 2-propanol, and dried under vacuum, thus affording 90.2 g of crude product (0.05% impurity V, 2.47% impurity III, wherein P is benzyl). The crude product was recrystallized from 2-propanol (0.810 L) and the obtained purified solid was dried under vacuum at 45° C.

Yield: 81.07 g (70.6%)

DSC: Sharp endotherm with an onset at 124.36° C., endotherm peak at 131.85° C.

HPLC: 99.9% (not detected (n.d) impurity V, 0.08% impurity III, wherein P is benzyl)

### Example 4: Preparation of benzyl (R)-(1-(carbamoyloxy)-3-phenylpropan-2-yl) Carbamate (Compound of Formula II Wherein P is -Cbz)

To a stirred suspension of benzyl (R)-(1-hydroxy-3-phenylpropan-2-yl) carbamate (100 g, 0.350 mol) and sodium cyanate (30.0 g, 0.461 mol) in 2.5 L of dichloromethane at 3-5° C., methane sulfonic acid (68.3 mL, 1.052 mmol) were added dropwise for 1 h. Once the addition was completed, the temperature was adjusted at 7-8° C. and the mixture was allowed to react for 4 h. One portion of sodium cyanate (5.03 g, 0.077 mol) was added to the suspension and the reaction mixture was stirred for 4 h. Another portion of sodium cyanate (5.03 g, 0.077 mol) was added to the mixture, the temperature was adjusted to 19-21° C. and the reaction mixture was stirred for 12 h. The reaction was quenched by addition of water (970 mL) and then pH adjusted to 10-10.5, with 20% aqueous NaOH solution, and then maintained under 10° C. affording a biphasic solution. The organic phase was washed with water (2.6 L) and the solvent partially distilled. Then 1.6 L of 2-propanol were added and distillation continued until all the methylene chloride was removed. Then the solution was cooled to 45° C. and seeded. After 30 minutes, temperature was adjusted to 30° C. and maintained for 4 h. The suspension was cooled to 15° C. and stirred for 1 h. Then the mixture was filtered off and the solid was washed with cold 2-propanol (n.d. impurity V, 0.79% impurity III, wherein P is benzyl). The crude product was recrystallized with 2-propanol and dried under vacuum at 45° C.

Yield: 80.8 g (70.2%)

HPLC: 99.9% (n.d. impurity V, 0.07% impurity III, wherein P is benzyl).

### Comparative example 1. Synthesis of (R)-2-amino-3-phenylpropyl carbamate from (R)-2-amino-3-phenylpropan-1-ol

In a dry round bottom flask dichloromethane (100 mL) was charged followed by addition of (R)-2-amino-3-phenylpropan-1-ol (5 g) and sodium cyanate (2.8 equiv.). The reaction mixture was cooled to about 0-5° C. and methane sulfonic acid (3.5 equiv.) was slowly added to the reaction mass. After completion of the addition of methane sulfonic acid, the temperature of the mixture was raised to about 20-30° C. and stirred for about 3 hours. After completion of the reaction, the mixture was cooled to 0-5° C. and 20% aqueous NaOH solution was added while maintaining the temperature below 5° C. The aqueous and organic layers were separated and the aqueous phase was extracted with dichloromethane. The combined organic phase was washed with water and brine and dried over sodium sulfate overnight followed by filtration. The filtrate was concentrated under vacuum to obtain solriamfetol as an oil.

Yield: 72%

HPLC: 80.3% (9.5% R-2-amino-3-phenylpropan-1-ol)

### Comparative example 2. Synthesis of (R)-2-amino-3-phenylpropyl carbamate from (R)-2-amino-3-phenylpropan-1-ol

To a stirred solution of (R)-2-amino-3-phenylpropan-1-ol (25 g, 0.330 mol) of in 500 mL of methylene chloride at 0-5° C., methane sulfonic acid was added dropwise (32.2 mL, 496 mmol) during 30 min. To the resulting mixture were added sodium cyanate 22.2 g (331 mol) in 6 portions maintaining the temperature at 0-5° C. The resulting mixture was stirred at 0-5° C. for 3 hours, and at room temperature overnight. After completion of the reaction, the reaction mass was cooled to about 0-5° C. and water was added (250 mL) followed by 20% aqueous NaOH solution to adjust the pH at 10.5. The aqueous and organic layers were separated and the aqueous phase was extracted with dichloromethane (2×125 mL). The combined organic phase was washed with 1% aqueous NaOH solution (125 mL), brine (75 mL) and water (75 mL), and dried over sodium sulfate overnight followed by filtration. The filtrate was concentrated under vacuum to obtain solriamfetol as an oil.

Yield: 76%

HPLC: 92.9% (1.9% 2R-2-amino-3-phenylpropan-1-ol)

### Example 5. Synthesis of (R)-2-amino-3-phenylpropyl carbamate hydrochloride

To a solution of benzyl (R)-(1-(carbamoyloxy)-3-phenylpropan-2-yl) carbamate (18.0 g, 54.816 mmol) in 400 mL of ethyl acetate were added 0.9 g of 10% (50% wet) Pd/C. The mixture was hydrogenated at 25-27° C. and 1 bar for 4 h. After inertization, 0.2 L of 2-propanol were added. The catalyst was filtered off and the clear solution was concentrated. The resulting crude was dissolved in 126 mL of 2-propanol (IPA) and cooled to 3-7° C. Then, trimethylchlorosilane (11.0 mL, 86.7 mmol) was added and the suspension was stirred for 4 h. The solid was filtered off, washed with cold 2-propanol and dried under vacuum at 45° C.

Yield: 11.57 g (91.5%)

HPLC: 99.9% (0.02% impurity A, n.d. % impurity B)

### Example 6. Synthesis of (R)-2-amino-3-phenylpropyl carbamate hydrochloride

To a solution of benzyl (R)-(1-(carbamoyloxy)-3-phenylpropan-2-yl) carbamate (25.0 g, 76.1 mmol) in 425 mL of tetrahydrofuran were added 1.0 g of 10% (50 wet) Pd/C. The mixture was hydrogenated at 30-31° C. and 1 bar for 4 h. After inertization the catalyst was removed by filtration and washed with tetrahydrofuran. The solvent was partially distilled and then 2-propanol (400 mL) were added. Distillation was continued until 350 ml of solvent were removed. This process was repeated twice. The resulting solution was cooled at 3-7° C. and then trimethylchlorosilane (46.2 mL, 364.0 mmol) was added and the suspension was stirred for 4 h. The solid was filtered off, washed with cold 2-propanol and dried under vacuum at 45° C.

Yield: 50.05 g (95.0%)

HPLC: 99.9% (0.05% impurity A, 0.01% impurity B)

1. A process for preparing (R)-2-amino-3-phenylpropyl carbamate, compound of formula (I), or a pharmaceutically acceptable acid addition salt thereof 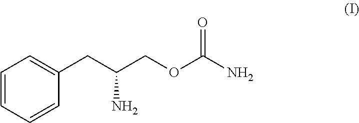 [SMILES: NC(=O)OC[C@H](N)Cc1ccccc1]
the process comprising the steps of:
a) providing a mixture comprising nitrogen-protected (R)-2-amino-3-phenylpropan-1-ol, compound of formula (III) 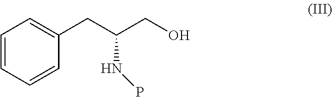
wherein P is a N-protecting group selected from carbobenzyloxy (Cbz), p-methoxybenzyl (PMB), and 3,4-dimethoxybenzyl (DMPM), and
an alkali metal cyanate in an amount of 1.0 to 1.8 molar equivalents with respect to compound of formula (III), in the presence of a solvent,
b) adding an acid with a pKa measured in water at 25° C. lower than 1.0 in an amount of 1.5 to 3.5 molar equivalents with respect to the alkali metal cyanate to the mixture of step (a), at a temperature from 0° C. to 10° C., to provide O-carbamoyl-N-protected-(R)-2-amino-3-phenylpropan-1-ol, compound of formula (II), wherein P is as defined above, 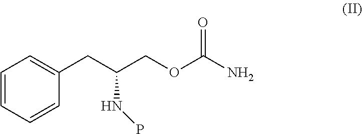
c) adding at least one portion of an alkali metal cyanate wherein each portion is used in an amount of 0.1 to 0.3 molar equivalents with respect to compound of formula (III) to the mixture of step (b), at a temperature from 0° C. to 10° C.,
d) removing the N-protecting group from the compound of formula (II) to give O-carbamoyl-(R)-2-amino-3-phenylpropan-1-ol, compound of formula (I), 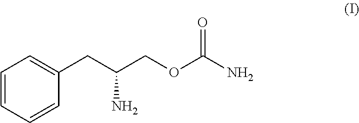 [SMILES: NC(=O)OC[C@H](N)Cc1ccccc1]
e) optionally, converting the compound of formula (I) into a pharmaceutically acceptable acid addition salt thereof.

2. The process according to claim 1, wherein the alkali metal cyanate of steps (a) and (c) is selected from the group consisting of sodium cyanate and potassium cyanate, and mixtures thereof.

3. The process according to claim 1, wherein the solvent of step (a) is a halogenated solvent selected from dichloromethane, 1,2-dichloroethane, chloroform, and chlorobenzene, and mixtures thereof.

4. The process according to claim 1, wherein the acid used in step (b) is selected from the group consisting of hydrochloric acid, hydrobromic acid and methane sulfonic acid, and mixtures thereof.

5. The process according to claim 1, wherein the mixture of step (c) is stirred at a temperature of from 0° C. to 10° C. for at least about 2 hours.

6. The process according to claim 1, wherein the resulting mixture of step (c) is further stirred at a temperature of from 15° C. to 25° C. for about 6 hours.

7. The process according to claim 1, comprising adding water and an alkali metal hydroxide to adjust the pH between 9 to 11, at a temperature from 0° C. to 10° C., to the mixture obtained after step (c) and before carrying out step (d).

8. The process according to claim 1, wherein compound of formula II obtained after step (c) is isolated and purified by crystallization from an alcohol selected from methanol, ethanol, isopropanol and 1-propanol, and mixtures thereof.

9. The process according to claim 1, wherein the N-protecting group of compound of formula II is removed in step (d) through hydrogenolysis in the presence of a catalyst and a solvent.

10. The process according to claim 9, wherein the catalyst is palladium on activated carbon (Pd/C) in an amount of 1% to 10% w/w with respect to the compound of formula II wherein P is as defined in claim 1.

11. The process according to claim 9, wherein the solvent in step (d) is selected from an ether, an alcohol and an ester solvent.

12. The process according to claim 1, wherein step (e) is carried out and compound of formula I is converted into the hydrochloride acid addition salt.

13. The process according to claim 12, comprising treating the compound of formula I with trimethylsilyl chloride in the presence of an alcohol selected from methanol, ethanol, 2-propanol or isopropanol and 1-propanol, and mixtures thereof.

14. The process according to claim 9, wherein steps (d) and (e) are carried out in a consecutive manner, wherein compound (I) thus obtained is not isolated.

15. The process according to claim 1, wherein the compound of formula (III), wherein P is as in claim 1, is prepared by a process which comprises the steps of: 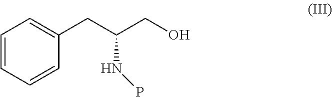
i. providing (R)-2-amino-3-phenylpropan-1-ol in a mixture of a ketone solvent and water in a ratio of 6:1 to 4:1,
ii. adding an inorganic base selected from an alkali metal carbonates and bicarbonates,
iii. adding a N-protecting group selected from the group consisting of benzyl-(2,5-dioxopyrrolidin-1-yl) carbonate, phenylchloroformate, p-methoxybenzyl (PMB), and 3,4-dimethoxybenzyl (DMPM), and
iv. optionally, isolating the corresponding N-protected-(R)-2-amino-3-phenylpropan-1-ol, compound of formula (III).

16. Hydrochloride acid addition salt of (R)-2-amino-3-phenylpropyl carbamate obtainable by the process according to claim 1 having simultaneously (R)-2-amino-3-phenylpropan-1-ol (impurity A) and the (R)-(2-amino-3-phenylpropyl)-N-carbamoylcarbamate (impurity B) in an amount below 0.15% w/w as measured by HPLC method B which is as follows:
Column: YMC-Pack ODS AQ; octadecyl silane silica gel for chromatography (5 μm) size 4.6
mm×25 cm
Flow: 1.2 mL/min
Wavelength: 210 nm
Column Temperature: 25° C.
Injection volume: 5 μL
Mobile phase A: 10 mM KH₂PO₄; pH=3.0 (H₃PO₄)
Mobile phase B: acetonitrile
Elution: Gradient (Table 2)
TABLE 2

| Time (min) | Mobile phase A (% V/V) | Mobile phase B (% V/V) |
|---|---|---|
| 0-10 | 90→77 | 10→23 |
| 10-20 | 77→25 | 23→75 |
| 20-25 | 25 | 75 |
| 25-25.10 | 25→90 | 75→10 |

Analysis time: 25.10 min
Retention time (RRT):
Impurity (A): 0.89
Impurity (B): 2.07
Benzyl (R)-(1-(carbamoyloxy)-3-phenylpropan-2-yl) carbamate: 3.10.

17. The process according to claim 2, wherein the alkali metal cyanate of steps (a) and (c) is sodium cyanate.

18. The process according to claim 1, wherein the solvent of step (a) is dichloromethane.

19. The process according to claim 4, wherein the acid used in step (b) is methane sulfonic acid.

20. The process according to claim 11, wherein the ether solvent when present is selected from the group consisting of diethyl ether, diisopropylether, diphenylether, tetrahydrofuran, 2-methyltetrahydrofuran and 1,4-dioxane, the alcohol solvent when present is selected from the group consisting of methanol, ethanol, isopropanol and 1-propanol, and the ester solvent when present is selected from the group consisting of methyl acetate, ethyl acetate and isopropyl acetate.
